(19) United States
(12) Patent Application Publication
(10) Pub. No.: US 2023/0279446 A1
(43) Pub. Date: Sep. 7, 2023

(54) METHOD FOR CONTROLLING MICROBIAL GROWTH IN AN ETHANOL FERMENTATION SYSTEM

(71) Applicant: HYDRITE CHEMICAL CO., Brookfield, WI (US)

(72) Inventors: Scott K. Cumming, Sun Prairie, WI (US); Matthew Kasper, Oak Creek, WI (US)

(21) Appl. No.: 18/112,681

(22) Filed: Feb. 22, 2023

Related U.S. Application Data
(60) Provisional application No. 63/312,924, filed on Feb. 23, 2022.

(51) Int. Cl. C12P 7/06; C12N 1/16; C12N 1/22; A01N 59/00; A01P 1/00; A23K 10/38

(52) U.S. Cl. CPC C12P 7/065; C12N 1/16; C12N 1/22; A01N 59/00; A01P 1/00; A23K 10/38; C12N 2500/05; C12N 2500/60

(57) ABSTRACT

A method for controlling microbial growth in an ethanol fermentation system is disclosed wherein the method comprises: (a) adding a biocide including a peroxy acid (e.g., peracetic acid) into a fermentable medium, wherein the biocide is essentially free of chelating agents; and (b) fermenting the fermentable medium with yeast to produce a fermented medium including ethanol. The method may further comprise: (c) distilling the fermented medium to separate at least a portion of the ethanol from solids in the fermented medium; and (d) producing a distillers grain product from the solids. By using a biocide essentially free of chelating agents, the method does not introduce undesirable chelating compounds into the co-product non-fermentable solids of ethanol production that are processed into distillers grain products.

### CROSS-REFERENCES TO RELATED APPLICATIONS

This application claims priority to U.S. Patent Application No. 63/312,924 filed Feb. 23, 2022.

### STATEMENT REGARDING FEDERALLY SPONSORED RESEARCH

Not Applicable.

### BACKGROUND OF THE INVENTION

### 1. Field of the Invention

The invention relates to methods for controlling microbial growth in an ethanol fermentation system. More particularly, the invention relates to methods for controlling microbial growth in an ethanol fermentation system wherein the methods do not introduce undesirable compounds present in biocides into the co-product solids of ethanol production that are processed into animal feed or animal feed additives.

### 2. Description of the Related Art

Over the past decades, significant attention has been given to the production of ethanol for use as an alternative fuel. Ethanol not only burns cleaner than fossil fuels, but also can be produced by fermentation using a wide variety of starch containing raw materials such as corn, which are renewable resources. Starch-based ethanol production generally includes converting a starchy feedstock into a fermentable medium including fermentable sugar, and adding yeast to the fermentable medium to ferment the sugar into ethanol. Ethanol is recovered by subjecting the fermented medium to distillation.

A problem in the ethanol industry is that fermentation process equipment and/or the fermentable medium can become contaminated with bacteria that reduce production yields. As a result, the efficiency of ethanol fermentation is significantly limited by other micro-organisms contaminating the process, such as lactic acid bacteria and acetic acid bacteria (e.g., yield loss bacterium). Contaminating bacteria compete for sugar supply with the yeast, resulting in a decrease in ethanol production. In addition, the contaminating bacteria can decrease the pH conditions which further inhibit the growth of ethanol-producing yeast. Biocides have been used as a means for controlling unwanted yield loss bacterium in fermentation plants.

A valuable co-product of distillation in ethanol production is solids containing proteins, fibers, and oils, which may be processed to produce distillers dried grains with solubles (“DDGS”). DDGS have a longer shelf life than normal corn and soybean meal, making DDGS a valuable food source to livestock and poultry producers. The high energy, mid-protein and high digestible phosphorus content of DDGS make it an attractive partial replacement for some of the more expensive and traditional energy (corn), protein (soybean), and phosphorus (monocalcium or dicalcium phosphate) used in animal feeds. Unfortunately, the use of biocides in ethanol fermentation methods can result in the incidental dosing of undesirable compounds present in biocides to such animals because certain compounds present in biocides survive through the ethanol distillation process and remain in the solids that are processed to produce DDGS. There is public opposition to such incidental dosing of certain undesirable compounds present in biocides into animal feeds.

Thus, there exists a need for improved methods for controlling microbial growth in an ethanol fermentation system wherein the methods do not introduce undesirable compounds present in biocides into the co-product solids of ethanol production that are processed into animal feed or animal feed additives.

### SUMMARY OF THE INVENTION

The present invention addresses the foregoing needs by providing improved methods for controlling microbial growth in an ethanol fermentation system.

In one aspect, the invention provides a method for controlling microbial growth in an ethanol fermentation system. The method comprises: (a) adding a biocide including a peroxy acid into a fermentable medium, wherein the biocide is essentially free of chelating agents; and (b) fermenting the fermentable medium with yeast to produce a fermented medium including ethanol.

In one embodiment of the method, the peroxy acid has a formula R¹CO₃H, where R¹ is selected from C₁ to C₁₈ alkyl. In another embodiment of the method, the peroxy acid has a formula R¹CO₃H, where R¹ is selected from C₁ to C₈ alkyl. In another embodiment of the method, the peroxy acid comprises peracetic acid.

In one embodiment of the method, step (a) comprises reacting a peroxide source with a carboxylic acid to form the peroxy acid. In one embodiment of the method, the peroxide source is hydrogen peroxide, and the carboxylic acid is acetic acid. In one embodiment of the method, the peroxide source and the carboxylic acid are reacted in the fermentable medium.

In one embodiment of the method, step (a) comprises adding the biocide including the peroxy acid into the fermentable medium such that a concentration of the peroxy acid in the fermentable medium is in a range of 1 ppm to 500 ppm. In another embodiment of the method, step (a) comprises adding the biocide including the peroxy acid into the fermentable medium such that a concentration of the peroxy acid in the fermentable medium is in a range of 2 ppm to 100 ppm. In another embodiment of the method, step (a) comprises adding the biocide including the peroxy acid into the fermentable medium such that a concentration of the peroxy acid in the fermentable medium is in a range of 2 ppm to 50 ppm. In another embodiment of the method, step (a) comprises adding the biocide including the peroxy acid into the fermentable medium such that a concentration of the peroxy acid in the fermentable medium is in a range of 2 ppm to 10 ppm.

In one embodiment of the method, step (a) comprises adding the biocide including the peroxy acid into the fermentable medium such that a pH in the fermentable medium is in a range of 4 to 6. In another embodiment of the method, step (a) comprises adding the biocide including the peroxy acid into the fermentable medium such that a pH in the fermentable medium is in a range of 3 to 7. In another embodiment of the method, step (a) comprises adding the biocide including the peroxy acid into the fermentable medium such that a pH in the fermentable medium is in a range of 4 to 8.

One embodiment of the method further comprises: (c) determining a concentration of the peroxy acid in the fermentable medium; and (d) adding an additional amount of the biocide including the peroxy acid into the fermentable medium when the concentration falls below a predetermined value.

One embodiment of the method further comprises: (c) sensing a measurable physical property of the fermentable medium; (d) generating a physical property signal corresponding to the measurable physical property, the physical property signal correlating to a concentration of the peroxy acid in the fermentable medium; (e) transmitting the physical property signal to a controller; and (f) when the concentration falls below a predetermined value stored in the controller, providing a control signal from the controller to open a supply valve in fluid communication with a source of the biocide including the peroxy acid and the fermentable medium thereby adding an additional amount of the biocide including the peroxy acid into the fermentable medium. In one embodiment of the method, the measurable physical property is selected from the group consisting of pH, conductivity, and oxidation reduction potential.

In one embodiment of the method, step (a) comprises adding the biocide including the peroxy acid into the fermentable medium in a cooker of the ethanol production system. In one embodiment of the method, step (a) comprises adding the biocide including the peroxy acid into the fermentable medium in a conduit that is positioned between a cooker and a liquefaction tank of the ethanol production system. In one embodiment of the method, step (a) comprises adding the biocide including the peroxy acid into the fermentable medium in a liquefaction tank of the ethanol production system. In one embodiment of the method, step (a) comprises adding the biocide including the peroxy acid into the fermentable medium in a conduit that is positioned between a liquefaction tank and a heat exchanger that is positioned between the liquefaction tank and a fermentation tank of the ethanol production system. In one embodiment of the method, step (a) comprises adding the biocide including the peroxy acid into the fermentable medium in a heat exchanger that is positioned between a liquefaction tank and a fermentation tank of the ethanol production system.

In one embodiment of the method, step (a) comprises adding the biocide including the peroxy acid into the fermentable medium in a conduit that is positioned between a first heat exchanger and a second heat exchanger, the first heat exchanger and the second heat exchanger being positioned between a liquefaction tank and a fermentation tank of the ethanol production system. In one embodiment of the method, step (a) comprises adding the biocide including the peroxy acid into the fermentable medium in a conduit that is positioned between a heat exchanger and a fermentation tank, the heat exchanger being positioned between a liquefaction tank and the fermentation tank of the ethanol production system. In one embodiment of the method, step (a) comprises adding the biocide including the peroxy acid into the fermentable medium in a fermentation tank of the ethanol production system. In one embodiment of the method, step (a) comprises adding the biocide including the peroxy acid into the fermentable medium in a fermentation cooler of the ethanol production system.

In one embodiment of the method, the biocide including the peroxy acid is added into the fermentable medium as a 1 wt. % to 35 wt. % aqueous solution of the peroxy acid. In one embodiment of the method, the biocide including the peroxy acid is added into the fermentable medium as a 1 wt. % to 25 wt. % aqueous solution of the peroxy acid. In one embodiment of the method, the biocide including the peroxy acid is added into the fermentable medium as a 1 wt. % to 15 wt. % aqueous solution of the peroxy acid. In one embodiment of the method, the biocide including the peroxy acid is added into the fermentable medium as a 1 wt. % to 10 wt. % aqueous solution of the peroxy acid.

In one embodiment of the method, the biocide includes an additional organic acid. The additional organic acid can be selected from the group consisting of lactic acid, citric acid, acetic acid, formic acid, oxalic acid, uric acid, malic acid, tartaric acid, benzoic acid, malonic acid, maleic acid, fumaric acid, succinic acid, gluconic acid, glutaric acid, and mixtures thereof. In one embodiment of the method, the additional organic acid is present in the biocide at 0.1 wt. % to 25 wt. % by total weight of the biocide.

In one embodiment of the method, the biocide includes an inorganic acid. The inorganic acid can be selected from the group consisting of hydrochloric acid, sulfuric acid, phosphoric acid, nitric acid, sulfamic acid, boric acid, hydrofluoric acid, hydrobromic acid, perchloric acid, hydroiodic acid, and mixtures thereof. In one embodiment of the method, the inorganic acid is present in the biocide at 0.1 wt. % to 25 wt. % by total weight of the biocide.

One embodiment of the method further comprises: (c) distilling the fermented medium to separate at least a portion of the ethanol from solids in the fermented medium; and (d) producing a distillers grain product from the solids. In one embodiment of the method, the distillers grain product is selected from the group consisting of wet distillers grain, distillers dried grains, and distillers dried grains with solubles.

In another aspect, the invention provides a method for producing a distillers grain product. The method comprises: (a) adding a biocide including a peroxy acid into a fermentable medium, wherein the biocide is essentially free of chelating agents; (b) fermenting the fermentable medium with yeast to produce a fermented medium including ethanol; (c) distilling the fermented medium to separate at least a portion of the ethanol from solids in the fermented medium; and (d) producing a distillers grain product from the solids.

In one embodiment of the method, the distillers grain product is wet distillers grain. In another embodiment of the method, the distillers grain product is distillers dried grains. In another embodiment of the method, the distillers grain product is distillers dried grains with solubles.

In one embodiment of the method, the peroxy acid has a formula R¹CO₃H, where R¹ is selected from C₁ to C₁₈ alkyl. In another embodiment of the method, the peroxy acid has a formula R¹CO₃H, where R¹ is selected from C₁ to C₈ alkyl. In another embodiment of the method, the peroxy acid comprises peracetic acid.

In the methods of the invention, the biocide including the peroxy acid (e.g., peracetic acid) is essentially free of chelating agents. By using a biocide essentially free of chelating agents, the method of the invention does not introduce undesirable chelating compounds into the co-product non-fermentable solids of ethanol production that are processed into wet distillers grain, or distillers dried grains, or distillers dried grains with solubles.

These and other features, aspects, and advantages of the present invention will become better understood upon consideration of the following detailed description, drawing, and appended claims.

### BRIEF DESCRIPTION OF THE DRAWINGS

FIG. 1 is a process flow diagram showing one version of a method according to the invention for controlling microbial growth in an ethanol fermentation system.

### DETAILED DESCRIPTION OF THE INVENTION

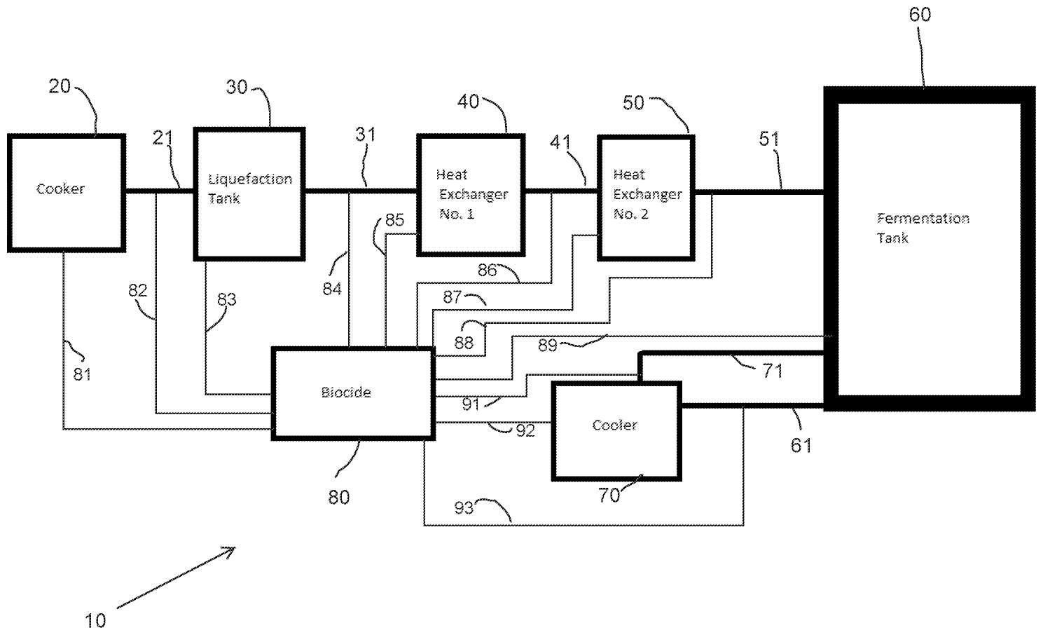

Referring now to FIG. 1, an ethanol production system 10 according to the invention is shown. Grain (e.g., corn) from a storage bin is ground into coarse flour that can be cooked and liquefied in a cooker 20. The cooked and liquefied mixture from the cooker 20 is pumped through a conduit 21 to a liquefaction tank 30. The mixture is held in the liquefaction tank 20 to give one or more enzymes (e.g., an amylase enzyme) time to break down the starch into fermentable sugars. The fermentable medium including fermentable sugars is pumped via a conduit 31 to a first heat exchanger 40, where the fermentable medium is cooled from a temperature of 185° F. to 130° F. The fermentable medium including fermentable sugars is pumped from the first heat exchanger 40 via a conduit 41 to a second heat exchanger 50 where the temperature is lowered to approximately 90° F. Once the temperature has been sufficiently lowered, the fermentable medium is directed via a conduit 51 to a fermentation tank 60. In the fermentation tank 60, yeast (e.g., Saccharomyces cerevisiae) ferments the fermentable sugars in the fermentable medium to produce ethanol. A recycle stream is withdrawn from the fermentation tank and is directed via a conduit 61 to a fermentation cooler 70 where the recycle stream is cooled and then returned to the fermentation tank 60 via a conduit 71.

In the ethanol production system 10, a biocide including a peroxy acid is stored in a reservoir 80. The biocide including a peroxy acid can be delivered from the reservoir 80 via any combination of the conduits 81, 82, 83, 84, 85, 86, 87, 88, 89, 91, 92, and 93 to various points in the ethanol production system 10. As shown in FIG. 1, the conduit 81 can deliver biocide including a peroxy acid from the reservoir 80 to the cooker 20; the conduit 82 can deliver biocide including a peroxy acid from reservoir 80 to the conduit 21; the conduit 83 can deliver biocide including a peroxy acid from the reservoir 80 to the liquefaction tank 30; the conduit 84 can deliver biocide including a peroxy acid from the reservoir 80 to the conduit 31; the conduit 85 can deliver biocide including a peroxy acid from the reservoir 80 to the first heat exchanger 40; the conduit 86 can deliver biocide including a peroxy acid from the reservoir 80 to the conduit 41; the conduit 87 can deliver biocide including a peroxy acid from the reservoir 80 to the second heat exchanger 50; the conduit 88 can deliver biocide including a peroxy acid from the reservoir 80 to the conduit 51; the conduit 89 can deliver biocide including a peroxy acid from the reservoir 80 to the fermentation tank 60; the conduit 91 can deliver biocide including a peroxy acid from the reservoir 80 to the conduit 71; the conduit 92 can deliver biocide including a peroxy acid from the reservoir 80 to the fermentation cooler 70; and the conduit 93 can deliver biocide including a peroxy acid from the reservoir 80 to the conduit 61. In a particularly advantageous version of the invention, the conduit 86 delivers biocide including a peroxy acid from the reservoir 80 to the conduit 41 between the first heat exchanger 40 and the second heat exchanger 50. In the conduit 41, the fermentable medium is approximately 130° F. which improves mixing of the biocide in the fermentable medium before the temperature of the fermentable medium is lowered to approximately 90° F. in the second heat exchanger 50.

The addition of a biocide including a peroxy acid via any combination of the conduits 81, 82, 83, 84, 85, 86, 87, 88, 89, 91, 92, and 93 can be continuous, substantially continuous, intermittent, cyclic, batch, or any combination thereof. Treatment with the biocide can be repeated any desired number of times, and treatments can be separated by constant or variable time periods. The rate of addition of a biocide including a peroxy acid can be constant or variable.

The fermented medium from the fermentation tank 60 can be pumped into a distillation unit, such as a multi-column distillation system that uses the differences in the boiling points of ethanol and water to boil off and separate the ethanol. The residue from this distillation process is called whole stillage, which contains non-fermentable solids and water. The whole stillage can be centrifuged for separation into thin stillage and a solids containing fraction that can be processed into distillers grains. At least some of the thin stillage can be sent through an evaporation system where it can be concentrated into syrup that can be mixed back in with the solids containing fraction to provide wet distillers grain (WDG), which comprises residual grain solids prior to drying and at least a portion of the process syrup. WDG can be animal feed grade, and can be used soon after it is produced. Alternatively, WDG is sent through a drying system to remove moisture and extend its shelf life. The resulting distillers dried grains with solubles (DDGS) is commonly used as a high-protein ingredient in animal feed, such as feed for cattle, swine, poultry, and fish. In another alternative, the solids containing fraction can be dried to produce distillers dried grains (DDG), which includes dried residual grain solids, and can be animal feed grade.

A peroxy acid included in the biocide used in the method of the invention can have a formula R¹CO₃H, where R¹ is selected from C₁ to C₁₈ alkyl, or the peroxy acid can have a formula R¹CO₃H, where R¹ is selected from C₁ to C₈ alkyl. In one version of the method, the biocide including a peroxy acid is added into the fermentable medium as an aqueous solution having 1 wt. % to 35 wt. % of the peroxy acid. In another version of the method, the biocide including a peroxy acid is added into the fermentable medium as an aqueous solution having 1 wt. % to 25 wt. % of the peroxy acid. In another version of the method, the biocide including a peroxy acid is added into the fermentable medium as an aqueous solution having 1 wt. % to 15 wt. % of the peroxy acid. In another version of the method, the biocide including a peroxy acid is added into the fermentable medium as an aqueous solution having 1 wt. % to 10 wt. % of the peroxy acid.

In one non-limiting example embodiment, the peroxy acid included in the biocide is peracetic acid. Chemically, the term “peracetic acid” describes two substances. “Pure” peracetic acid has the chemical formula CH₃CO₃H. Anhydrous peracetic acid explodes violently upon heating. In contrast, aqueous solutions of peracetic acid as sold commercially are created by combining aqueous mixtures of two substances: acetic acid and hydrogen peroxide. At cool temperatures, acetic acid and hydrogen peroxide react over a few days to form an equilibrium aqueous solution containing peracetic acid, acetic acid and hydrogen peroxide. Adding a mineral acid catalyst accelerates the reaction. Peracetic acid is an unstable oxidizing agent and therefore, conventional peracetic acid solutions contain a chelating agent such as etidronic acid (1-hydroxyethylidene-1, 1-diphosphonic acid or HEDP) or dipicolinic acid (pyridine-2,6-dicarboxylic acid or DPA) to slow the rate of oxidation or decomposition of the peracetic acid.

In the method of the invention, the biocide including the peroxy acid (e.g., peracetic acid) is essentially free of chelating agents. As used herein, “essentially free of chelating agents” means that chelating agents are not added to the biocide and that chelating agents are not added to the reactants used to prepare the biocide, but chelating agents may be present as an impurity or undesired contaminant in the biocide. By using a biocide essentially free of chelating agents, the method of the invention does not introduce undesirable chelating compounds into the co-product non-fermentable solids of ethanol production that are processed into wet distillers grain (WDG), or distillers dried grains (DDG), or distillers dried grains with solubles (DDGS).

In one version of the method, the biocide including peracetic acid is added into the fermentable medium as an aqueous solution including peracetic acid, acetic acid and hydrogen peroxide wherein the peracetic acid is present at 1 wt. % to 35 wt. % in the aqueous solution including peracetic acid, acetic acid and hydrogen peroxide. In another version of the method, the biocide including peracetic acid is added into the fermentable medium as an aqueous solution including peracetic acid, acetic acid and hydrogen peroxide wherein the peracetic acid is present at 1 wt. % to 25 wt. % in the aqueous solution including peracetic acid, acetic acid and hydrogen peroxide. In another version of the method, the biocide including peracetic acid is added into the fermentable medium as an aqueous solution including peracetic acid, acetic acid and hydrogen peroxide wherein the peracetic acid is present at 1 wt. % to 15 wt. % in the aqueous solution including peracetic acid, acetic acid and hydrogen peroxide. In another version of the method, the biocide including peracetic acid is added into the fermentable medium as an aqueous solution including peracetic acid, acetic acid and hydrogen peroxide wherein the peracetic acid is present at 1 wt. % to 10 wt. % in the aqueous solution including peracetic acid, acetic acid and hydrogen peroxide. In another version of the method, the biocide including peracetic acid is added into the fermentable medium as an aqueous solution including peracetic acid, acetic acid and hydrogen peroxide wherein the peracetic acid is present at 1 wt. % to 5 wt. % in the aqueous solution including peracetic acid, acetic acid and hydrogen peroxide.

Alternatively, the biocide including a peroxy acid may be prepared by mixing a peroxide source, such as hydrogen peroxide, and an acid which is a precursor of a chosen peroxy acid. The mixing may occur before the biocide including a peroxy acid is added into the ethanol production system 10; or the mixing may occur after a peroxide source, such as hydrogen peroxide, and a precursor acid which is a precursor of the biocide including a peroxy acid are added into a vessel or conduit of the ethanol production system 10. For example, the biocide including a peroxy acid may be prepared by reacting a peroxide source with a carboxylic acid to form the peroxy acid. The peroxide source and the carboxylic acid may be reacted in the fermentable medium in a vessel or conduit of the ethanol production system 10. In one non-limiting example embodiment, the peroxide source is hydrogen peroxide, and the carboxylic acid is acetic acid.

In one version of the method, the biocide including a peroxy acid is added into the fermentable medium of the ethanol production system 10 such that a concentration of the peroxy acid in the fermentable medium of the ethanol production system is in a range of 1 ppm to 500 ppm. In another non-limiting example version of the method, the biocide including a peroxy acid is added into the fermentable medium of the ethanol production system 10 such that a concentration of the peroxy acid in the fermentable medium of the ethanol production system is in a range of 2 ppm to 100 ppm. In another non-limiting example version of the method, the biocide including a peroxy acid is added into the fermentable medium of the ethanol production system 10 such that a concentration of the peroxy acid in the fermentable medium of the ethanol production system is in a range of 2 ppm to 50 ppm. In another non-limiting example version of the method, the biocide including a peroxy acid is added into the fermentable medium of the ethanol production system 10 such that a concentration of the peroxy acid in the fermentable medium of the ethanol production system is in a range of 2 ppm to 10 ppm.

One non-limiting example biocide for use in the method is an aqueous solution including 4.9 wt. % peracetic acid, 5-30% wt. % hydrogen peroxide, 2-20% wt. % acetic acid, and balance water to 100% by weight. Another non-limiting example biocide for use in the method is an aqueous solution including 5.9 wt. % peracetic acid, 15-35% wt. % hydrogen peroxide, 2-20% wt. % acetic acid, and balance water to 100% by weight. Yet another non-limiting example biocide for use in the method is an aqueous solution including 15 wt. % peracetic acid, 5-30% wt. % hydrogen peroxide, 10-30% wt. % acetic acid, and balance water to 100% by weight. Still another non-limiting example biocide for use in the method is an aqueous solution including 22 wt. % peracetic acid, 5-30% wt. % hydrogen peroxide, 40-60% wt. % acetic acid, and balance water to 100% by weight.

In one non-limiting example version of the method, the biocide including a peroxy acid is added into the fermentable medium of the ethanol production system such that a pH in the fermentable medium of the ethanol production system is in a range of 4 to 6. In another non-limiting example version of the method, the biocide including a peroxy acid is added into the fermentable medium of the ethanol production system such that a pH in the fermentable medium of the ethanol production system is in a range of 3 to 7. In another non-limiting example version of the method, the biocide including a peroxy acid is added into the fermentable medium of the ethanol production system such that a pH in the fermentable medium of the ethanol production system is in a range of 2 to 8.

In one non-limiting example version of the method, the biocide includes an additional organic acid. The additional organic acid can be selected from the group consisting of lactic acid, citric acid, acetic acid, formic acid, oxalic acid, uric acid, malic acid, tartaric acid, benzoic acid, malonic acid, maleic acid, fumaric acid, succinic acid, gluconic acid, glutaric acid, and mixtures thereof. In one version of the method, the additional organic acid can be selected from the group consisting of lactic acid, citric acid, and mixtures thereof. The additional organic acid can be present in the biocide at 0.1 wt. % to 25 wt. % by total weight of the biocide. The additional organic acid can be present in the biocide at 0.1 wt. % to 15 wt. % by total weight of the biocide. The additional organic acid can be present in the biocide at 0.1 wt. % to 10 wt. % by total weight of the biocide. The additional organic acid can be present in the biocide at 0.1 wt. % to 5 wt. % by total weight of the biocide.

In one non-limiting example version of the method, the biocide includes an inorganic acid. The inorganic acid can be selected from the group consisting of hydrochloric acid, sulfuric acid, phosphoric acid, nitric acid, sulfamic acid, boric acid, hydrofluoric acid, hydrobromic acid, perchloric acid, hydroiodic acid, and mixtures thereof. The inorganic acid can be selected from the group consisting of hydrochloric acid, sulfuric acid, phosphoric acid, nitric acid, and mixtures thereof. The inorganic acid can be present in the biocide at 0.1 wt. % to 25 wt. % by total weight of the biocide. The inorganic acid can be present in the biocide at 0.1 wt. % to 15 wt. % by total weight of the biocide. The inorganic acid can be present in the biocide at 0.1 wt. % to 10 wt. % by total weight of the biocide. The inorganic acid can be present in the biocide at 0.1 wt. % to 5 wt. % by total weight of the biocide.

Automated control of the addition of the biocide including a peroxy acid to the fermentable medium of the ethanol production system is possible. A sensor can be placed in the fermentable medium in a vessel or conduit of the ethanol production system such that the fermentable medium contacts the sensor. The sensor measures a physical property of the fermentable medium. As used herein, a physical property or a measurable physical property is a property of matter that can be measured or observed without resulting in a change in the composition and identity of a substance. Non-limiting examples of physical properties that can be measured in the sensor include pH, conductivity, oxidation reduction potential, concentration, and density. Sensors are commercially available for measuring these physical properties of the fermentable medium.

It is contemplated that direct feedback from the sensor can be sent to a programmable logic controller to provide opening and closing times for various valves that control addition of the biocide including a peroxy acid to a vessel or conduit of the ethanol production system 10 via any combination of the conduits 81, 82, 83, 84, 85, 86, 87, 88, 89, 91, 92, and 93. For example, in one version of the method of the invention, the controller can determine a concentration of the peroxy acid in the fermentable medium of the ethanol production system using signals from the sensor, and an additional amount of the biocide including the peroxy acid can be added into the fermentable medium of the ethanol production system by opening a valve when the concentration falls below a predetermined value. In another version of the method of the invention, the sensor is used to sense a measurable physical property (e.g., pH, conductivity, and oxidation reduction potential) of the fermentable medium of the ethanol production system; the sensor generates a physical property signal corresponding to the measurable physical property wherein the physical property signal correlates to a concentration of the peroxy acid in the fermentable medium in a vessel or conduit of the ethanol production system; the sensor transmits the physical property signal to the controller; and when the concentration falls below a predetermined value stored in the controller, the controller provides a control signal to open a supply valve in fluid communication with a source of the biocide including a peroxy acid and the ethanol production system 10 thereby adding an additional amount of the biocide including the peroxy acid into the fermentable medium in a vessel or conduit of the ethanol production system.

Use of the biocide in the method of the invention encompasses ethanol production using any carbohydrate or starch material that is a source of fermentable sugar, either as a direct source of fermentable sugar, or as a material which can provide fermentable sugar by degradation or conversion of the original or an intermediate starch, cellulose, or polysaccharide component thereof. Examples of suitable sources of feedstock materials are agricultural crops, such as grains (e.g., corn, wheat, sorghum, barley, rice, rye, oats), sugar cane, sugar beets, molasses, potatoes, carrots, cassava, rhubarb, and parsnips. Ethanol can be produced by fermentation with methods of the present invention using other starchy feedstock materials such as biomass, for example, wood chips, sawdust, switchgrass, corn stover, corn cobs, straw, grain hulls, as well as recycled paper and waste paper materials, or any combinations thereof. Additional feedstock materials can include fruits and/or fruit juices (e.g., grapes, plums, berries, apples, pears, cherries), refined sugar (e.g., sucrose), honey, tree sap, or any combinations thereof. It is understood in the industry that these and/or other different feedstocks for ethanol fermentation may have different ethanol yields, such as due to different starch contents and compositions, and different co-products. The methods of the present invention using the biocide for bacteria control in ethanol production can be used without limitation with respect to the starchy feedstock.

Thus, the invention provides methods for controlling microbial growth in an ethanol fermentation system. The method of the invention reduces bacteria count of bacteria that reduce production yields of ethanol. Thus, the method increases yield of ethanol. In addition, the methods do not introduce undesirable compounds present in biocides into the co-product non-fermentable solids of ethanol production that are processed into animal feed or animal feed additives.

In light of the principles and example embodiments described and illustrated herein, it will be recognized that the example embodiments can be modified in arrangement and detail without departing from such principles. Also, the foregoing discussion has focused on particular embodiments, but other configurations are also contemplated. In particular, even though expressions such as “in one embodiment”, “in another embodiment,” or the like are used herein, these phrases are meant to generally reference embodiment possibilities, and are not intended to limit the invention to particular embodiment configurations. As used herein, these terms may reference the same or different embodiments that are combinable into other embodiments. As a rule, any embodiment referenced herein is freely combinable with any one or more of the other embodiments referenced herein, and any number of features of different embodiments are combinable with one another, unless indicated otherwise.

Although the present invention has been described in detail with reference to certain embodiments, one skilled in the art will appreciate that the present invention can be practiced by other than the described embodiments, which have been presented for purposes of illustration and not of limitation. Therefore, the scope of the appended claims should not be limited to the description of the embodiments contained herein.

1. A method for controlling microbial growth in an ethanol fermentation system, the method comprising:
(a) adding a biocide including a peroxy acid into a fermentable medium, wherein the biocide is essentially free of chelating agents; and
(b) fermenting the fermentable medium with yeast to produce a fermented medium including ethanol.

2. (canceled)

3. The method of claim 1 wherein:
the peroxy acid has a formula R¹CO₃H, where R¹ is selected from C₁ to C₈ alkyl.

4. The method of claim 1 wherein:
the peroxy acid comprises peracetic acid.

5. The method of claim 1 wherein:
step (a) comprises reacting a peroxide source with a carboxylic acid to form the peroxy acid,
the peroxide source is hydrogen peroxide,
the carboxylic acid is acetic acid, and
the peroxide source and the carboxylic acid are reacted in the fermentable medium.

6. (canceled)

7. (canceled)

8. (canceled)

9. (canceled)

10. (canceled)

11. (canceled)

12. (canceled)

13. (canceled)

14. The method of claim 1 wherein:
step (a) comprises adding the biocide including the peroxy acid into the fermentable medium such that a pH in the fermentable medium is in a range of 4 to 8.

15. The method claim 1 further comprising:
(c) determining a concentration of the peroxy acid in the fermentable medium and
(d) adding an additional amount of the biocide including the peroxy acid into the fermentable medium when the concentration falls below a predetermined value.

16. The method of 1 further comprising:
(c) sensing a measurable physical property of the fermentable medium;
(d) generating a physical property signal corresponding to the measurable physical property, the physical property signal correlating to a concentration of the peroxy acid in the fermentable medium;
(e) transmitting the physical property signal to a controller; and
(f) when the concentration falls below a predetermined value stored in the controller, providing a control signal from the controller to open a supply valve in fluid communication with a source of the biocide including the peroxy acid and the fermentable medium thereby adding an additional amount of the biocide including the peroxy acid into the fermentable medium.

17. (canceled)

18. The method of claim 1 wherein:
step (a) comprises adding the biocide including the peroxy acid into the fermentable medium in a cooker of the ethanol production system.

19. The method of claim 1 wherein:
step (a) comprises adding the biocide including the peroxy acid into the fermentable medium in a conduit that is positioned between a cooker and a liquefaction tank of the ethanol production system.

20. The method of claim 1 wherein:
step (a) comprises adding the biocide including the peroxy acid into the fermentable medium in a liquefaction tank of the ethanol production system.

21. The method of claim 1 wherein:
step (a) comprises adding the biocide including the peroxy acid into the fermentable medium in a conduit that is positioned between a liquefaction tank and a heat exchanger that is positioned between the liquefaction tank and a fermentation tank of the ethanol production system.

22. The method of claim 1 wherein:
step (a) comprises adding the biocide including the peroxy acid into the fermentable medium in a heat exchanger that is positioned between a liquefaction tank and a fermentation tank of the ethanol production system.

23. The method of claim 1 wherein:
step (a) comprises adding the biocide including the peroxy acid into the fermentable medium in a conduit that is positioned between a first heat exchanger and a second heat exchanger, the first heat exchanger and the second heat exchanger being positioned between a liquefaction tank and a fermentation tank of the ethanol production system.

24. The method of claim 1 wherein:
step (a) comprises adding the biocide including the peroxy acid into the fermentable medium in a conduit that is positioned between a heat exchanger and a fermentation tank, the heat exchanger being positioned between a liquefaction tank and the fermentation tank of the ethanol production system.

25. The method of claim 1 wherein:
step (a) comprises adding the biocide including the peroxy acid into the fermentable medium in a fermentation tank of the ethanol production system.

26. The method of claim 1 wherein:
step (a) comprises adding the biocide including the peroxy acid into the fermentable medium in a fermentation cooler of the ethanol production system.

27. The method of claim 1 wherein:
the biocide including the peroxy acid is added into the fermentable medium as a 1 wt. % to 35 wt. % aqueous solution of the peroxy acid.

28. (canceled)

29. (canceled)

30. (canceled)

31. The method of claim 1 wherein:
the biocide includes an additional organic acid.

32. (canceled)

33. (canceled)

34. The method of claim 1 wherein:
the biocide includes an inorganic acid.

35. (canceled)

36. (canceled)

37. The method of 1 further comprising:
(c) distilling the fermented medium to separate at least a portion of the ethanol from solids in the fermented medium; and
(d) producing a distillers grain product from the solids.

38. (canceled)

39. A method for producing a distillers grain product, the method comprising:
(a) adding a biocide including a peroxy acid into a fermentable medium, wherein the biocide is essentially free of chelating agents;
(b) fermenting the fermentable medium with yeast to produce a fermented medium including ethanol;
(c) distilling the fermented medium to separate at least a portion of the ethanol from solids in the fermented medium; and
(d) producing a distillers grain product from the solids.

40. (canceled)

41. (canceled)

42. (canceled)

43. (canceled)

44. (canceled)

45. (canceled)
